(19) United States
(12) Patent Application Publication
(10) Pub. No.: US 2025/0050089 A1
(43) Pub. Date: Feb. 13, 2025

(54) FLEXIBLE PLUG FOR FLUID FLOW PATH

(71) Applicants: BAXTER INTERNATIONAL INC., Deerfield, IL (US); BAXTER HEALTHCARE SA, Glattpark (Opfikon) (CH)

(72) Inventors: Robert Wilkie SCHELL, Deerfield, IL (US); Rajkumar KARTHIKEYAN, Banagalore Karnataka (IN); Bharath Nuggehalli SOMASHIYANDAN, Banagalore Karnataka (IN)

(21) Appl. No.: 18/720,466

(22) PCT Filed: Dec. 12, 2022
(86) PCT No.: PCT/US2022/081339

§ 371 Date: Jun. 14, 2024

(30) Foreign Application Priority Data
Dec. 20, 2021 (IN) 202141059393

(51) Int. Cl. A61M 39/20

(52) U.S. Cl. CPC A61M 39/20

(57) ABSTRACT

A flexible plug for a medical fluid comprises a sealing member including a flexible cylindrical section that extends to a head; a rigid member holder secured to the sealing member; and a rigid sealing cap including a wall defining an opening, the head of the sealing member sealed within the opening to prevent the flow of medical fluid through the flexible plug. The flexible plug is sealed, e.g., solvent bonded, within a port tube, sleeve port or a Y-connector to form a flexible plug assembly. The user grasps and twists or torques the rigid member holder and the rigid sealing cap through the port tube, sleeve port or a Y-connector to unseal the head from the opening to allow the flow of medical fluid through the flexible plug. A method of manufacturing the flexible plug and associated assembly is also disclosed.

### BACKGROUND

The present disclosure relates generally to medical fluid treatments and in particular to medical fluid treatments using premade or bagged medical fluid.

Due to various causes, a person's renal system can fail. Renal failure produces several physiological derangements. It is no longer possible to balance water and minerals or to excrete daily metabolic load. Toxic end products of metabolism, such as, urea, creatinine, uric acid and others, may accumulate in a patient's blood and tissue.

Reduced kidney function and, above all, kidney failure is treated with dialysis. Dialysis removes waste, toxins, and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid or PD fluid, into a patient's peritoneal chamber via a catheter. The PD fluid comes into contact with the peritoneal membrane in the patient's peritoneal chamber. Waste, toxins, and excess water pass from the patient's bloodstream, through the capillaries in the peritoneal membrane, and into the PD fluid due to diffusion and osmosis, i.e., an osmotic gradient occurs across the membrane. An osmotic agent in the PD fluid provides the osmotic gradient. Used PD fluid is drained from the patient, removing waste, toxins, and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis, and continuous flow peritoneal dialysis (“CFPD”). CAPD is a manual dialysis treatment. Here, the patient manually connects an implanted catheter to a drain to allow used PD fluid to drain from the patient's peritoneal cavity. The patient then switches fluid communication so that the patient catheter communicates with a bag of fresh PD fluid to infuse the fresh PD fluid through the catheter and into the patient. The patient disconnects the catheter from the fresh PD fluid bag and allows the PD fluid to dwell within the patient's peritoneal cavity, wherein the transfer of waste, toxins and excess water takes place. After a dwell period, the patient repeats the manual dialysis procedure, for example, four times per day. Manual peritoneal dialysis requires a significant amount of time and effort from the patient, leaving ample room for improvement.

APD is similar to CAPD in that the dialysis treatment includes drain, fill, and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh PD fluid and to a fluid drain. APD machines pump fresh PD fluid from a dialysis fluid source, through the catheter and into the patient's peritoneal chamber. APD machines also allow for the PD fluid to dwell within the chamber and for the transfer of waste, toxins and excess water to take place. The source may include multiple liters of dialysis fluid, including several solution bags.

APD machines pump used PD fluid from the patient's peritoneal cavity, though the catheter, to drain. As with the manual process, several drain, fill and dwell cycles occur during dialysis. A “last fill” may occur at the end of the APD treatment. The last fill fluid may remain in the peritoneal chamber of the patient until the start of the next treatment, or may be manually emptied at some point during the day.

Any of the above treatment modalities may operate with premade, e.g., bagged, solutions. Bagged solutions are typical for any type of PD (CAPD or APD). A bagged solution may also be used for HD, especially HHD (see for example U.S. Pat. No. 8,029,454 assigned to the assignee of the present application). Continuous renal replacement therapy (“CRRT”) is an acute form of HD, HF or HDF and typically uses bagged dialysis fluids.

Premade, e.g., bagged, solutions for any of the above modalities are typically filled, capped, and then sterilized to maintain the medical fluid in a sterilized condition until use. There are different ways to access the sterilized solutions at the time of use. One way is to spike the connector at the time of use, establishing medical fluid flow between the bag and a use point, such as a patient or a disposable cassette. Another way, which is very common with PD, is to use a breakable frangible. The patient or caregiver bends and snaps open the breakable frangible to thereafter allow fluid flow.

Problems with particulate matter (“PM”) may be present with a connector that is punctured or a frangible that is broken. Small splinters or other type of PM may come free from the connector or frangible and be carried away in the flow of medical fluid. The PM may flow to an undesirable location, such as the patient or disposable cassette, and perhaps create an undesirable visual for the patient or caregiver if the PM is visible.

There is accordingly a need for an improved way to selectively allow medical fluid to flow from a storage bag to a use point.

### SUMMARY

The present disclosure involves the use of a flexible plug for use with a solution container or bag operable with any type of dialysis treatment including any type of peritoneal dialysis (“PD”) treatment, hemodialysis (“HD”) treatment, hemofiltration (“HF”) treatment, hemodiafiltration (“HDF”) treatment, or continuous renal replacement therapy (“CRRT”) treatment. It should be appreciated that the flexible plug may be used in any type of medical treatment having a bagged or otherwise stored medical fluid, which needs to be opened aseptically for use. The flexible plug may therefore be used additionally with any type of bagged medical infusion or intravenous fluid, saline, lactated ringers, etc.

The flexible plug in one embodiment includes three components, namely, a rigid sealing cap, a flexible sealing member, and a rigid member holder. Those three components are assembled and then placed within a port tube to form a flexible plug assembly. The port tube is attached at one end to the medical fluid container or bag. The other end of the port tube includes a connector for connecting to a mating connector of a line that leads from the container to a use point, e.g., a patient, a drain (for priming), a disposable pumping cassette, etc.

The rigid sealing cap and the rigid member holder may be formed, e.g., molded, from a thermoplastic, such as polyetherimide (“PEI”), polyethersulfone (“PES”), polyamide/nylon (“PA”), acrylonitrile butadiene styrene (“ABS”), polycarbonate (“PC”), polypropylene (“PP”), and polyvinylchloride (“PVC”). The flexible sealing member may be formed, e.g., molded from an elastomer, such as ethylene propylene diene monomer (“EPDM”) rubber, neoprene rubber, silicon rubber, thermo-plastic vulcunizates (“TPVs”) and thermos-plastic elastomers (“TPEs”). The port tube may be made of PVC or other suitable medically safe material.

The rigid member holder in one embodiment defines an annular seat that accepts a mating flange located at one end of the flexible sealing member. The member holder also defines a bore that extends from the annular seat through an end of the member holder. The flexible sealing member includes a flexible cylindrical section that extends from the mating flange, through the bore, where the inner diameter of the bore and the outer diameter of the flexible cylindrical section are virtually the same (due to the molding process used). The member holder therefore holds the flexible sealing member firmly via the seat and flange engagement. The member holder is also able to apply a torque to the flexible sealing member via the bore and flexible cylindrical section engagement. The member holder further includes a section extending from the annular seat, in the opposite direction from the bore. The extension section helps the user (patient or caregiver) to apply a twisting or torqueing force to the member holder and flexible sealing member.

The flexible cylindrical section of the flexible sealing member extends out from the bore of the member holder, through a lumen defined by rigid sealing cap. The inner diameter of the lumen of the sealing cap is larger than the outer diameter of the flexible cylindrical section, which allows the flexible cylindrical section to bend within the lumen. The sealing cap includes an endwall that in one embodiment defines a beveled opening. The flexible cylindrical section extends through the sealing cap lumen to the endwall of the sealing cap. The end of the flexible cylindrical section residing at the endwall includes or defines a head that is beveled or otherwise shaped so as to seal within the beveled opening of the endwall.

To open the flexible plug of the present disclosure, the patient or caregiver bends the port tube so as to angle the rigid member holder relative to the rigid sealing cap. Here the patient or caregiver may grasp the port tube at a location overlying the rigid sealing cap with one hand and grasp the port tube at a location overlying the extension section of the rigid member holder with their other hand to twist or torque the flexible plug so as to angle the rigid member holder relative to the rigid sealing cap. The action of angling the rigid member holder relative to the rigid sealing cap causes the flexible cylindrical section of the flexible sealing member to bend or buckle to thereby cause the beveled head of the flexible sealing member to angle away from and come at least partially free from the beveled opening of the endwall of the sealing cap. The beveled head of the flexible sealing member is partially pulled into the lumen defined of the rigid sealing cap, allowing static medical fluid pressure, which has operated previously to push the beveled head of the flexible sealing member against the beveled opening of the endwall of the sealing cap, to now push the flexible sealing member into the lumen defined of the rigid sealing cap. The beveled head of the flexible sealing member is now completely free from the beveled opening of the endwall of the sealing cap, allowing medical fluid to flow to the use point.

It is contemplated to assemble the flexible plug of the present disclosure (after rigid sealing cap, flexible sealing member, and rigid member, and port tube have been formed) in a plurality of steps. In a first step, the flexible sealing member is overmolded over the rigid member holder. In an alternative embodiment, 2K molding is performed instead of overmolding to secure the flexible sealing member in place relative to the rigid member holder. In a second step the rigid sealing cap is placed into registry with the secured flexible sealing member and rigid member holder. In a third step, the rigid sealing cap is inserted over the flexible cylindrical section of the flexible sealing member so that the rigid sealing cap abuts up against a mating end of rigid member holder.

In a fourth step, the beveled head of the flexible sealing member is sealed against the beveled opening of the endwall of the sealing cap. It is contemplated to perform the fourth step in one of a plurality of different ways. In one way, the end of the flexible sealing member is thermoformed after insertion of the sealing cap to form the beveled head of the flexible sealing member in place and sealed against the beveled opening of the endwall of the sealing cap. Here, the end of the flexible sealing member may extend out from the beveled opening of the endwall of the sealing cap initially to provide additional elastomeric material with which to form the beveled head of the flexible sealing member.

In a second way, the beveled head is formed separately and is for example adhered, e.g., solvent bonded, to the end of the flexible cylindrical section of the flexible sealing member. In either case, the bevels of the beveled head and the beveled opening may each be completely straight or provided with half-rounds. In one example, the flexible sealing member is molded with a beveled end to which a half-round head is adhered, and which is sealed within the opening of the endwall of the sealing cap, which is here a half-round opening instead of a beveled opening.

In a fifth assembly step, the connected rigid sealing cap, flexible sealing member, and rigid member holder of the flexible plug are inserted inside of the port tube so as to be press-fittingly connected in one embodiment. The press-fit prevents the connected rigid sealing cap, flexible sealing member, and rigid member holder of the flexible plug from sliding inside of the port tube and from allowing medical fluid from flowing around the outside of the flexible plug. The port tube may or may not be connected to the medical fluid container prior to performing the fifth assembly step. It should be appreciated that the port tube may alternatively be (or be sealed within) a sleeve port or a Y-connector.

In an alternative embodiment of the flexible plug, the flexible sealing member and the rigid member holder are largely the same, while rigid sealing cap is modified such that the beveled opening of the rigid sealing cap is provided instead at or near an entrance or proximal end to receive the beveled head of the flexible sealing member. The rigid sealing cap still provides a lumen, but the lumen is open in one embodiment at the distal end instead of providing the beveled opening, which is provided instead at or near an entrance or proximal end of the cap. The lumen is provided to give the rigid sealing cap some length to allow the patient or caregiver to grasp the cap from the outside of the port tube, and along with grasping the rigid member holder through the port tube, twist of torque the cap and the member holder relative to each other to bend the flexible sealing member, disrupting the beveled seal between the cap and the flexible sealing member, and allowing medical fluid to flow.

In light of the disclosure set forth herein, and without limiting the disclosure in any way, in a first aspect of the present disclosure, which may be combined with any other aspect, or portion thereof, a flexible plug for a medical fluid includes a sealing member including a flexible section that extends to a head; a rigid member holder secured to the sealing member; and a rigid sealing cap including a wall defining an opening, the head of the sealing member sealed within the opening to prevent a flow of medical fluid through the flexible plug.

In a second aspect of the present disclosure, which may be combined with any other aspect, or portion thereof, the rigid member abuts against the rigid sealing cap.

In a third aspect of the present disclosure, which may be combined with any other aspect, or portion thereof, the wall defining the opening is an endwall of the rigid sealing cap.

In a fourth aspect of the present disclosure, which may be combined with any other aspect, or portion thereof, the wall defining the opening is located at an inner portion of the rigid sealing cap.

In a fifth aspect of the present disclosure, which may be combined with any other aspect, or portion thereof, the opening is a beveled opening and the head of the sealing member is a beveled head.

In a sixth aspect of the present disclosure, which may be combined with any other aspect, or portion thereof, the opening includes at least one half-round and the head of the sealing member includes at least one mating half-round.

In a seventh aspect of the present disclosure, which may be combined with any other aspect, or portion thereof, the head is formed from the same material as the flexible section or is attached to the flexible section.

In an eighth aspect of the present disclosure, which may be combined with any other aspect, or portion thereof, the rigid member holder includes an annular seat that accepts a mating flange of the sealing member.

In a ninth aspect of the present disclosure, which may be combined with any other aspect, or portion thereof, the flexible section extends from the mating flange of the sealing member through a bore defined by the rigid member holder.

In a tenth aspect of the present disclosure, which may be combined with any other aspect, or portion thereof, the flexible section includes a flexible cylindrical section.

In an eleventh aspect of the present disclosure, which may be combined with any other aspect, or portion thereof, a flexible plug assembly for a medical fluid includes a sealing member including a flexible section that extends to a head: a rigid member holder secured to the sealing member: a rigid sealing cap including a wall defining an opening, the head of the sealing member sealed within the opening to prevent a flow of medical fluid through the flexible plug; and a port tube, sleeve port or Y-connector, at least one of the sealing member, rigid member and rigid sealing cap sealed within the port tube, sleeve port or Y-connector.

In a twelfth aspect of the present disclosure, which may be combined with any other aspect, or portion thereof, the port tube, sleeve port or Y-connector is flexible so as to allow a user to grasp and twist or torque the rigid member holder and the rigid sealing cap through the port tube, sleeve port or Y-connector to unseal the head from the opening and to allow the flow of medical fluid through the flexible plug.

In a thirteenth aspect of the present disclosure, which may be combined with any other aspect, or portion thereof, the rigid member abuts against the rigid sealing cap, the rigid member and rigid sealing cap sealed within the port tube, sleeve port or Y-connector.

In a fourteenth aspect of the present disclosure, which may be combined with any other aspect, or portion thereof, a method for unsealing a sealed medical fluid plug includes causing a head located at an end of a flexible section of a sealing member to seal within an opening of a rigid sealing cap, the sealing member held by a rigid member holder; and enabling the rigid sealing cap and the rigid member holder to be twisted or torqued relative to each other to unseal the head from the opening and allow a flow of medical fluid through the medical fluid plug.

In a fifteenth aspect of the present disclosure, which may be combined with any other aspect, or portion thereof, enabling the rigid sealing cap and the rigid member holder to be twisted or torqued relative to each other includes locating the rigid sealing cap and the rigid member holder within a flexible port tube, sleeve port or Y-connector.

In a sixteenth aspect of the present disclosure, which may be combined with any other aspect, or portion thereof, the method includes structuring the rigid member holder to have flow-through channels to allow the flow of medical fluid across the rigid member holder after unsealing the head from the opening.

In a seventeenth aspect of the present disclosure, which may be combined with any other aspect, or portion thereof, the method includes structuring the rigid sealing cap to allow the flexible section to flex within the rigid sealing cap to unseal the head from the opening when the rigid sealing cap and the rigid member holder are twisted or torqued relative to each other.

In an eighteenth aspect of the present disclosure, which may be combined with any other aspect, or portion thereof, a method for manufacturing a medical fluid plug includes overmolding or 2K molding a flexible sealing member and a rigid member holder; inserting a rigid sealing cap over a flexible section of the flexible sealing member, the rigid sealing cap including a wall defining an opening; and forming or attaching a head to an end of the flexible section of the flexible sealing member so that the head seals the opening in the wall of the rigid sealing cap.

In a nineteenth aspect of the present disclosure, which may be combined with any other aspect, or portion thereof, the method includes thermoforming the head at the end of the flexible section of the flexible sealing member so that the head seals the opening in the wall of the rigid sealing cap.

In a twentieth aspect of the present disclosure, which may be combined with any other aspect, or portion thereof, the method includes adhering the head to the end of the flexible section of the flexible sealing member so that the head seals the opening in the wall of the rigid sealing cap.

In a twenty-first aspect of the present disclosure, which may be combined with any other aspect, or portion thereof, the method includes sealing at least the rigid sealing cap within a port tube, sleeve port or Y-connector to form a medical fluid plug assembly.

In a twenty-second aspect of the present disclosure, which may be combined with any other aspect, or portion thereof, any of the features, functionality and alternatives described in connection with any one or more of FIGS. 1 to 11 may be combined with any of the features, functionality and alternatives described in connection with any other of FIGS. 1 to 11.

In light of the above aspects and the present disclosure set forth herein, it is accordingly an advantage of the present disclosure to provide a flexible medical fluid plug that generates no particulate matter (“PM”) during activation.

It is another advantage of the present disclosure to provide a flexible medical fluid plug that reduces the amount of force needed to obtain medical fluid flow.

It is a further advantage of the present disclosure to provide a flexible medical fluid plug that mitigates potential patient dexterity issues.

It is yet another advantage of the present disclosure to provide a flexible medical fluid plug, the actuation of which creates a one way medical fluid flow direction.

It is yet a further advantage of the present disclosure to provide a flexible medical fluid plug that helps to prevent microbial contaminate and to maintain a sterile fluid path.

It is still another advantage of the present disclosure to provide a flexible medical fluid plug that requires no outside tool for fluid flow activation.

It is still a further advantage of the present disclosure to provide a flexible medical fluid plug that may be produced using advanced molding techniques, such as 2k injection molding.

Yet another advantage of the present disclosure is to provide a flexible medical fluid plug that is joined/connected using varieties of PVC or non-PVC materials.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is an exploded perspective view of one embodiment of a flexible plug of the present disclosure.

FIG. 2 is a sectioned perspective view of one embodiment of a flexible plug of the present disclosure located within a port tube and showing one possible medical fluid flowpath after the plug has been actuated.

FIG. 3 is a sectioned elevation view of a flexible plug of the present disclosure located within a port tube and blocking medical fluid flow.

FIG. 4A is a sectioned elevation view of a flexible plug of the present disclosure during actuation.

FIG. 4B is a sectioned elevation view of a flexible plug of the present disclosure relaxed after the actuation of FIG. 4A.

FIG. 5 is a sectioned elevation view of a flexible plug of the present disclosure after the actuation of FIGS. 4A and 4B.

FIG. 6 is a sectioned perspective view of one embodiment of a flexible plug of the present disclosure showing one possible medical fluid flowpath when the plug is actuated.

FIGS. 7A to 7D illustrate alternative ways of forming a beveled (or half-rounded) head to beveled (or half-rounded) opening seal.

FIG. 8 is an exploded perspective view of an alternative embodiment of a flexible plug of the present disclosure.

FIG. 9 is a sectioned elevation view of the alternative flexible plug of FIG. 8.

FIG. 10 is a partially sectioned plan view of one embodiment for implementing any of the flexible plugs of the present disclosure in an automated peritoneal dialysis (“APD”) arrangement.

FIG. 11 is a partially sectioned plan view of one embodiment for implementing any of the flexible plugs of the present disclosure in a continuous ambulatory peritoneal dialysis (“CAPD”) arrangement.

### DETAILED DESCRIPTION

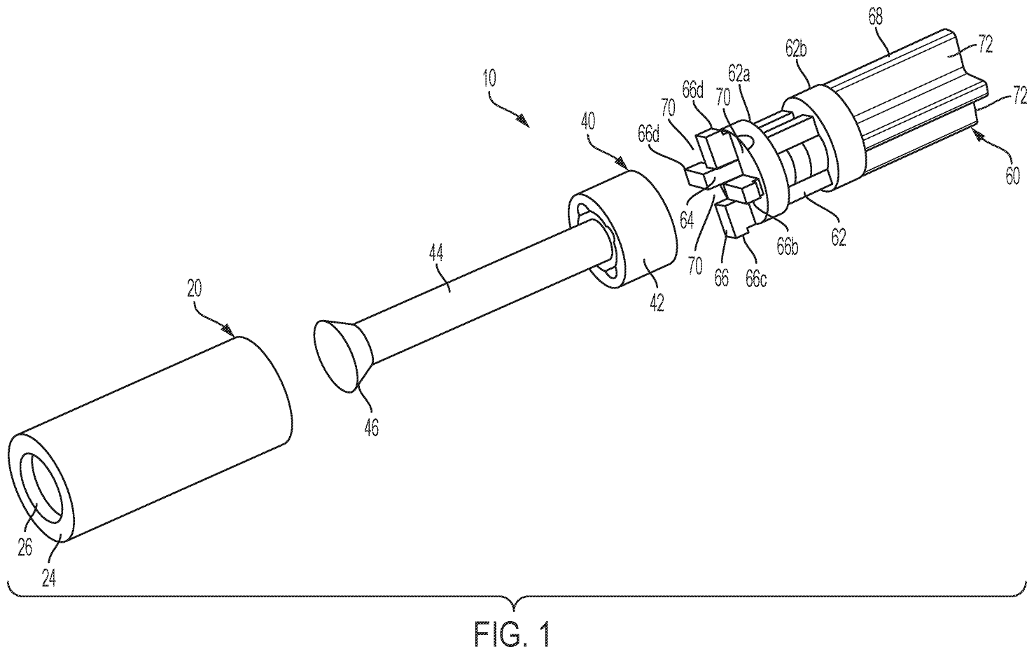

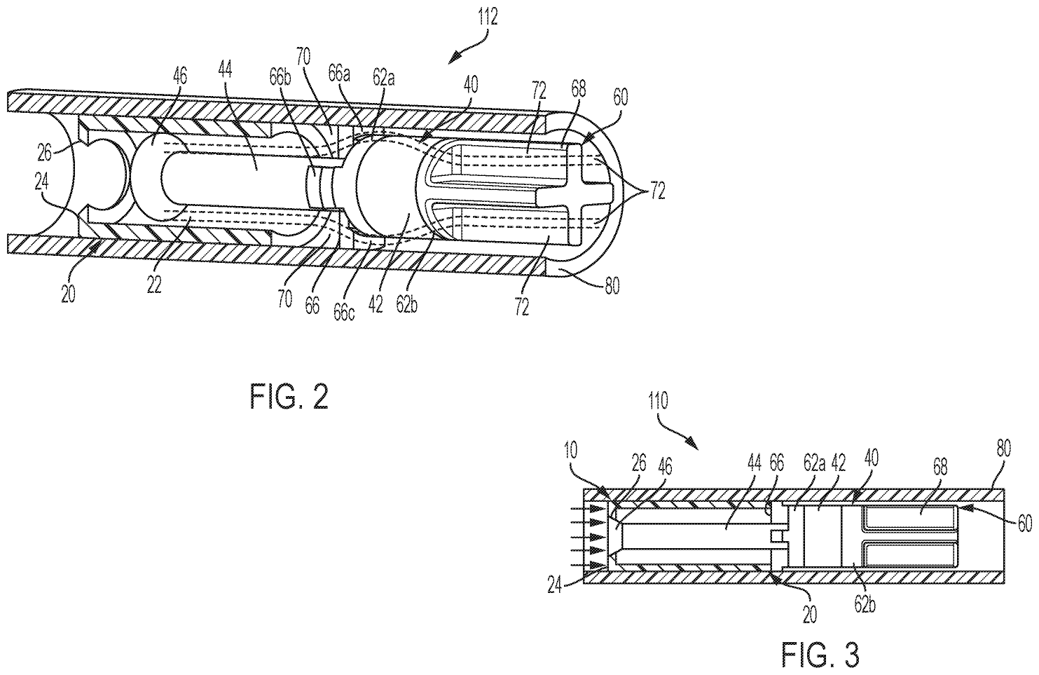

Referring now to the drawings and in particular to FIGS. 1 and 2, one embodiment for a flexible plug 10 for use with a medical solution container or bag is illustrated. The medical solution container or bag may be operable with any type of dialysis treatment including any type of peritoneal dialysis (“PD”) treatment, hemodialysis (“HD”) treatment, hemofiltration (“HF”) treatment, hemodiafiltration (“HDF”) treatment or continuous renal replacement therapy (“CRRT”) treatment. It should be appreciated that flexible plug 10 may be used in any type of medical treatment having a bagged or otherwise stored medical fluid, which needs to be opened aseptically for use. Flexible plug 10 may therefore be used additionally with any type of bagged medical infusion or intravenous fluid, saline, lactated ringers, etc.

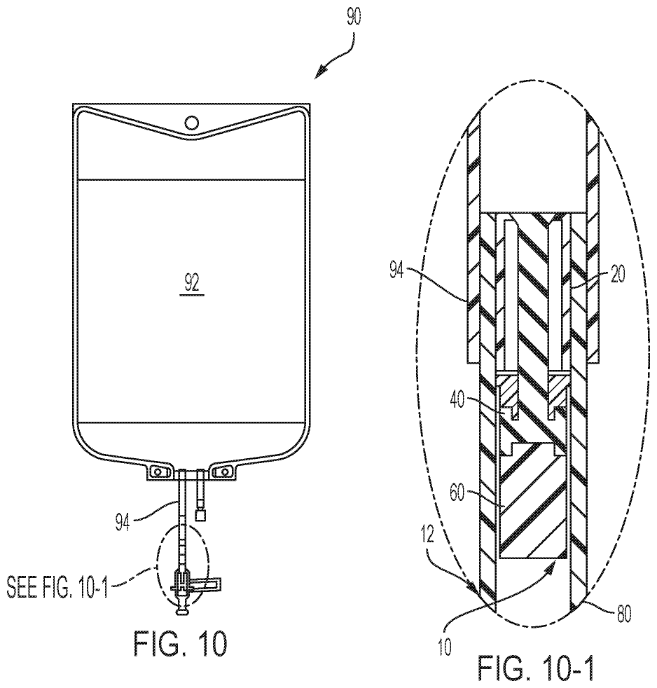

Flexible plug 10 in one embodiment includes three components, namely, a rigid sealing cap 20, a flexible sealing member 40, and a rigid member holder 60. Those three components are assembled and then placed within a port tube 80 to form a flexible plug assembly 12. Flexible plug assembly 12, including all components thereof, may be sterilized via any known method, such as steam, ethylene oxide, gamma radiation, electron-beam, and x-ray sterilization. Port tube 80 is in one embodiment attached at one end to the medical fluid container or bag (see FIG. 10). The other end of port tube 80 extends to a patient luer in one embodiment (see FIG. 10) for connecting to a mating connector of a line (not illustrated) that leads to a use point, e.g., a patient, a drain (for priming), a disposable pumping cassette, etc.

Rigid sealing cap 20 and rigid member holder 60 may be formed, e.g., molded, from a thermoplastic, such as polyetherimide (“PEI”), polyethersulfone (“PES”), polyamide/nylon (“PA”), acrylonitrile butadiene styrene (“ABS”), polycarbonate (“PC”), polypropylene (“PP”), and polyvinylchloride (“PVC”). Flexible sealing member 40 may be formed, e.g., molded from an elastomer, such as ethylene propylene diene monomer (“EPDM”) rubber, neoprene rubber, silicon rubber, thermo-plastic vulcunizates (“TPVs”) and thermos-plastic elastomers (“TPEs”). Port tube 80 may be made of PVC or other suitable medically safe material.

Rigid member holder 60 in one embodiment defines an annular seat 62 that accepts a mating flange 42 located at one end of flexible sealing member 40. Member holder 60 also defines a bore 64 that extends from annular seat 62 through an end 66 of member holder 60. Flexible sealing member 40 includes a flexible cylindrical section 44 that extends from mating flange 42, through bore 64, where the inner diameter of bore 64 and the outer diameter of flexible cylindrical section 44 are virtually the same (due to the molding process used). Member holder 60 therefore holds flexible sealing member 40 firmly via the engagement of seat 62 and flange 42. Member holder 60 is also able to apply a torque to flexible sealing member 40 via the engagement of bore 64 and flexible cylindrical section 44. Member holder 60 further includes a section 68 extending from annular seat 62, in the opposite direction from bore 64. Extension section 68 helps the user (patient or caregiver) to apply a twisting or torqueing force to member holder 60 and flexible sealing member 40.

Flexible cylindrical section 44 of flexible sealing member 40 extends out from bore 64 of member holder 60, through a lumen 22 defined by rigid sealing cap 20. The inner diameter of lumen 22 of sealing cap 20 is larger than the outer diameter of flexible cylindrical section 44, which allows flexible cylindrical section 44 to bend within lumen 22. Sealing cap 20 includes an endwall 24 that in one embodiment defines a beveled opening 26. Flexible cylindrical section 44 extends through sealing cap lumen 22 to endwall 24 of sealing cap 20. The end of flexible cylindrical section 44 residing at endwall 24 includes or defines a head 46 that is beveled or otherwise shaped so as to seal within beveled opening 26 of the endwall 24.

FIG. 3 illustrates that when flexible plug 10 is assembled, and sealed within port tube 80 to form a flexible plug assembly 12, fluid flow is blocked as indicated by the stoppage of the arrows. In FIG. 3, sealing cap 20 and port tube 80 are shown in cross-section to be able to see sealing member 40 and member holder 60. In the illustrated embodiment, the outer surface of sealing cap 20 is press-fitted and this fluidically sealed within port tube 80. Flexible sealing member 40 and member holder 60 are not press-fitted within port tube 80 in one embodiment so that they may move or translate relative to the captured sealing cap 20. Beveled head 46 is shown as being sealed within beveled opening 26 at endwall 24 of sealing cap 20. Fluid, as illustrated by the arrows, is therefore prevented from flowing around the outside of sealing cap 20 or through beveled opening 26 of sealing cap 20.

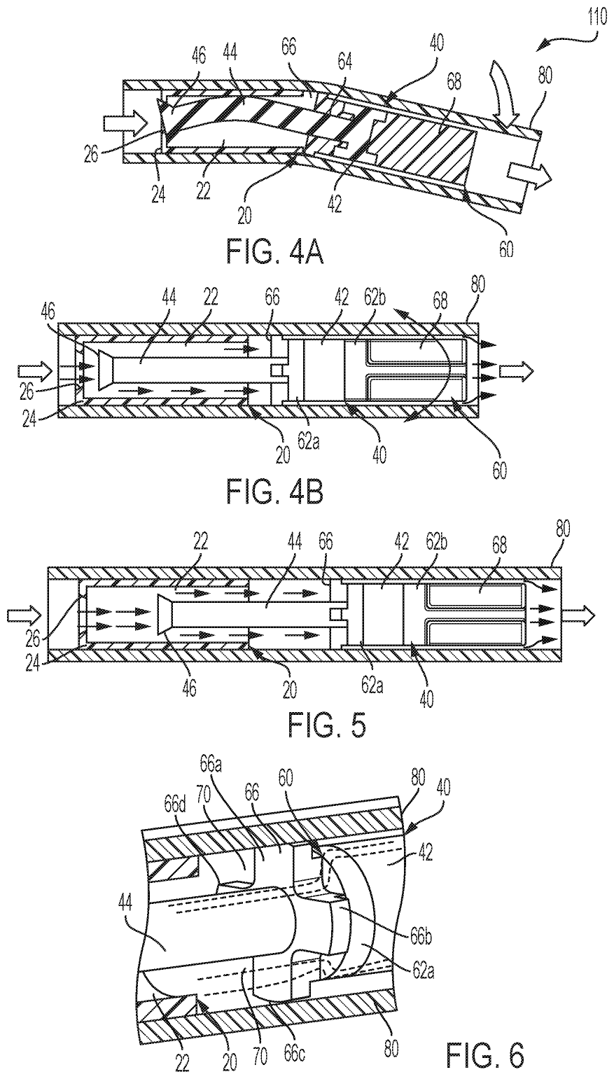

FIGS. 4A, 4B and 5 illustrate that to open flexible plug 10 of the present disclosure, the patient or caregiver bends, twists or torques port tube 80 so as to angle rigid member holder 60 relative to rigid sealing cap 20. Here, the patient or caregiver may grasp port tube 80 at a location overlying rigid sealing cap 20 with one hand and grasp port tube 80 at a location overlying extension section 68 of rigid member holder 60 with their other hand to twist or torque flexible plug 10, so as to angle rigid member holder 60 relative to rigid sealing cap 20. FIG. 4A illustrates that the action of angling rigid member holder 60 relative to rigid sealing cap 20 causes flexible cylindrical section 44 of flexible sealing member 40 to bend or buckle, to thereby cause beveled head 46 of flexible sealing member 40 to angle away from and come at least partially free from beveled opening 26 of endwall 24 of sealing cap 20. FIG. 4B illustrates that after relaxing flexible plug 10 after the twisting or torqueing in FIGS. 4A, beveled head 46 of flexible sealing member 40 is partially pulled into lumen 22 defined of rigid sealing cap 20, allowing static medical fluid pressure, which has operated previously to push beveled head 46 of flexible sealing member 40 against beveled opening 26 of endwall 24 of sealing cap 20, to now push the flexible sealing member 40 into lumen 22 defined of rigid sealing cap 20. Beveled head 46 of flexible sealing member 40 is now completely free from beveled opening 26 of endwall 24 of sealing cap 20, allowing medical fluid to flow to the use point as illustrated by the arrows in FIG. 5.

FIGS. 1, 2 and 6 illustrate that end 66 of rigid member holder 60 includes multiple, e.g., four, separate segments 66a to 66d, which define flow-through channels 70 between the segments. Also, the outer partial diameters of segments 66a to 66d are slightly larger than the diameters of cylindrical lengths 62a and 62 that border annular seat 62 (see also FIGS. 3, 4B and 5). The outermost diameter of extension section 68 is also smaller than partial diameters of segments 66a to 66d. Extension section 68 also defines flow-through channels 72. Thus when beveled head 46 of flexible sealing member 40 is torqued or twisted free from beveled opening 26 (FIG. 4A), and is translated away from endwall 24 of sealing cap 20 under fluid pressure (FIG. 5), any of the fluids described herein are able to flow (i) through beveled opening 26, (ii) through lumen 22, (iii) through flow-through channels 70, (iv) over cylindrical lengths 62a and 62, (v) through flow-through channels 72, and (vi) out to a desired destination through port tube 80, as generally indicated by the dashed flow lines in FIGS. 2 and 6.

Referring to FIGS. 1 and 3, it is contemplated to assemble flexible plug 10 of the present disclosure (after each of rigid sealing cap 20, flexible sealing member 40, and rigid member, and port tube 80 have been formed) in a plurality of steps. In a first step, flexible sealing member 40 is overmolded over rigid member holder 60 or vice versa as illustrated in FIG. 3. In an alternative embodiment, 2K molding is performed instead of overmolding to secure flexible sealing member 40 in place relative to rigid member holder 60. In a second step, rigid sealing cap 20 is placed into registry with the secured flexible sealing member 40 and rigid member holder 60. In a third step, rigid sealing cap 20 is inserted over flexible cylindrical section 44 of flexible sealing member 40, so that rigid sealing cap 20 abuts up against mating end 66 of rigid member holder 60. It should be appreciated that sealing cap 20 does not have to actually contact mating end 66 of rigid member holder 60 for flexible plug 10 for the plug to operate properly, however doing so may make the formation of beveled head 46 and the insertion of plug 10 into port tube 80 easier.

In a fourth step, beveled head 46 of flexible sealing member 40 is sealed against beveled opening 26 of endwall 24 of sealing cap 20. It is contemplated to perform the fourth step in one of a plurality of different ways. In one way as illustrated by FIG. 3, the end 48 of flexible sealing member 40 is thermoformed and compressed after insertion of sealing cap 20 to form the shape of beveled head 46 of flexible sealing member 40 in place and to seal head 46 against beveled opening 26 of endwall 24 of sealing cap 20. Here, the end 48 of flexible sealing member 40 may initially extend out from beveled opening 26 of sealing cap 20, and have the shape of flexible cylindrical section 44, to provide additional elastomeric material with which to form the shape of beveled head 46 of flexible sealing member 40 and to seal beveled head 46 to beveled opening 26.

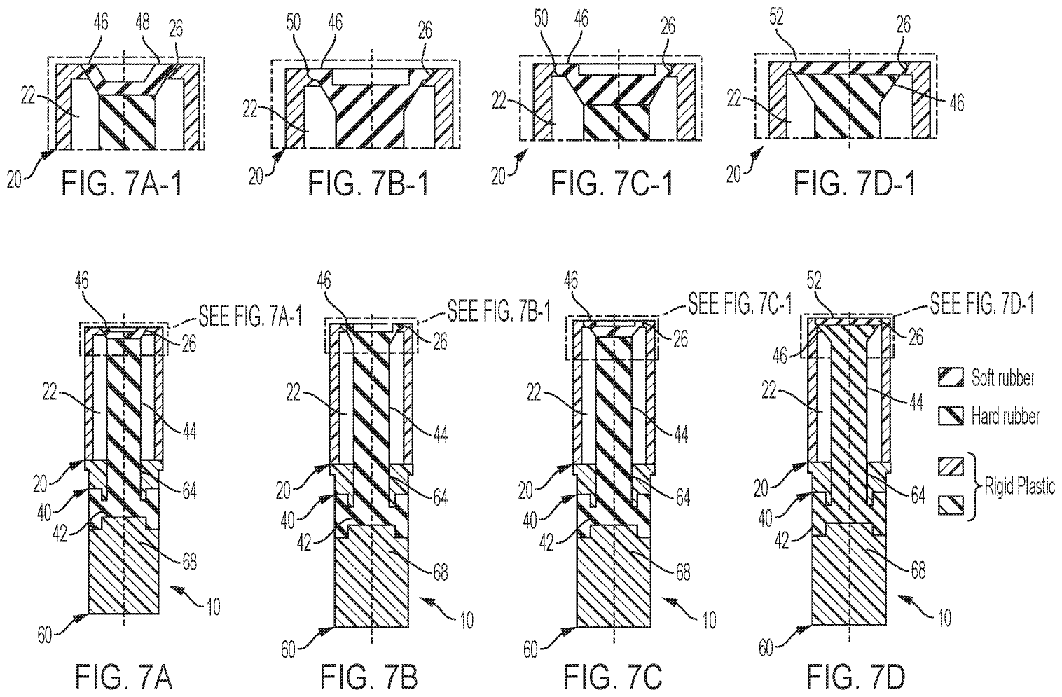

In a second way as illustrated by FIG. 7A, beveled head 46 is formed separately and is for example adhered, e.g., solvent bonded, to the end of flexible cylindrical section 44 of flexible sealing member 40, so as to seal within beveled opening 26. Here, beveled head 46 may be made of a soft hardness rubber, while cylindrical section 44 is made of a hard rubber. Another way to manufacture in FIG. 7A is to use a 3K molding technique, which may significantly reduce the manufacturing time.

In a third way as illustrated by FIG. 7B, beveled head 46 is molded with high hardness flexible cylindrical section 44. Here, the profile of beveled head 46 is provided with a half-round diameter 50, which sealingly snap-fits within beveled opening 26 at the endwall 24 of sealing cap 20.

In a fourth way as illustrated by FIG. 7C, beveled head 46 is formed with a half-round diameter 50 using a soft rubber and is then adhered, e.g., solvent bonded, to the end of flexible cylindrical section 44 of flexible sealing member 40 (made of hard rubber), so as to seal within beveled opening 26.

In a fifth way as illustrated by FIG. 7D, a half-rounded tip 52, e.g., made of soft rubber is formed separately and is adhered, e.g., solvent bonded, to beveled head 46, which is formed along with cylindrical section 44 from hard rubber. Half-rounded tip 52 is sealed, e.g., snap-fitted, into a like-shaped opening 26 formed in endwall 24 of sealing cap 20.

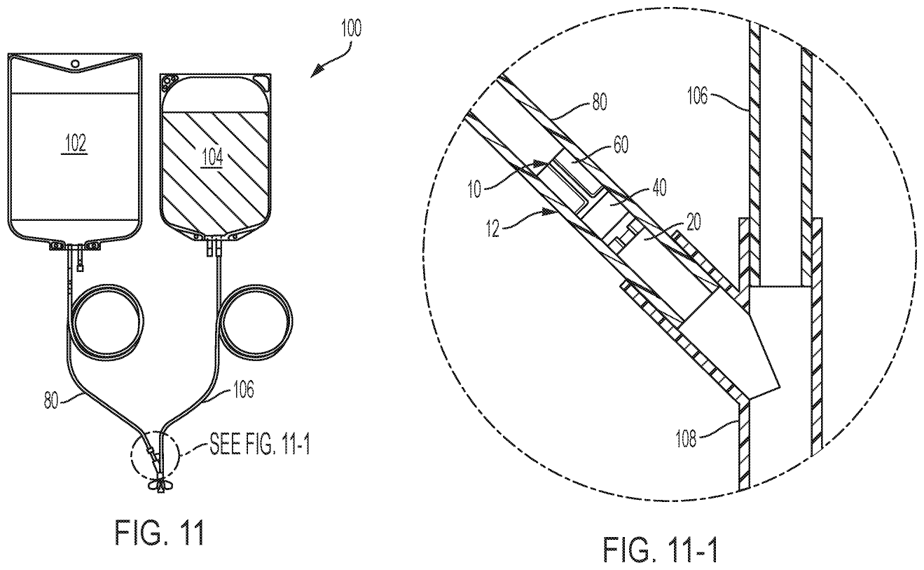

In a fifth assembly step, the connected rigid sealing cap 20, flexible sealing member 40, and rigid member holder 60 of flexible plug 10 are inserted inside of port tube 80, e.g., a flexible port tube, so as to be press-fittingly sealed in one embodiment. The press-fit is only be between port tube 80 and rigid sealing cap 20 in one embodiment, so that rigid sealing cap 20 is held fixed and flexible sealing member 40 with rigid member holder 60 can translate upon actuation for fluid flow. In any case, the press-fit prevents the connected rigid sealing cap 20, flexible sealing member 40, and rigid member holder 60 of flexible plug 10 from sliding inside of port tube 80 and from allowing medical fluid from flowing around the outside of flexible plug 10. Port tube 80 may or may not be connected to the medical fluid container prior to performing the fifth assembly step. It should be appreciated that port tube 80 may alternatively be (or be sealed within) a sleeve port or a Y-connector (see FIG. 11).

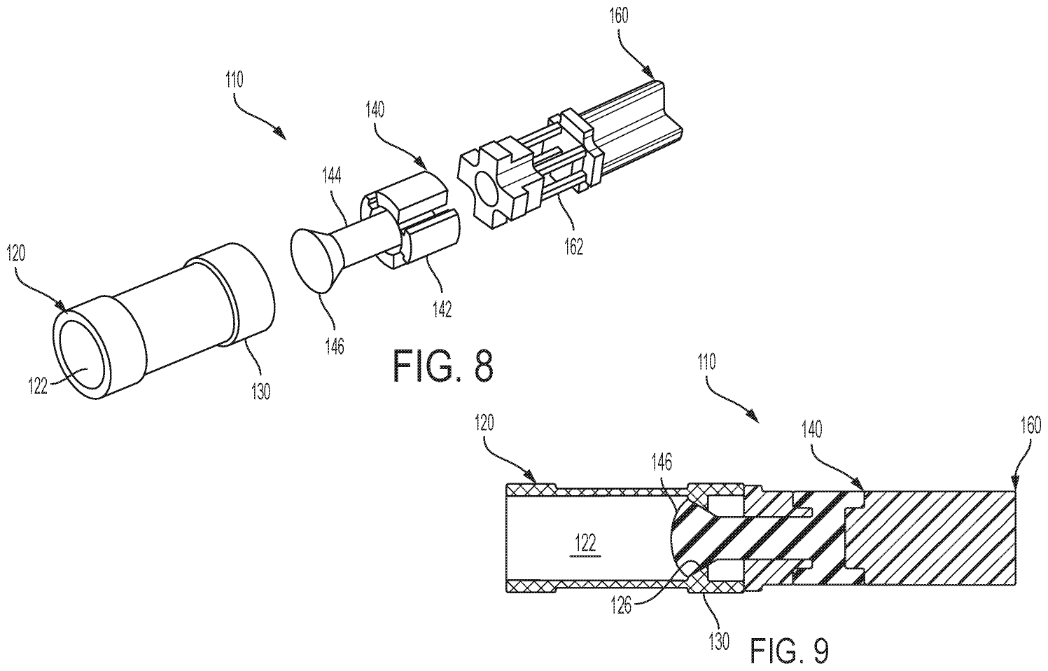

FIGS. 8 and 9 illustrate an alternative embodiment of flexible plug 110, wherein flexible sealing member 140 and rigid member holder 160 are largely the same as member 40 and holder 60, with mating flange 142 overmolded or 2K molded in place with annular seat 162. Rigid sealing cap 120 is modified such that beveled opening 126 of rigid sealing cap 120 is provided instead at or near an inner entrance or proximal end 130 of the cap to receive beveled head 146 (manufactured in any of the manners described above) of flexible sealing member 140. Rigid sealing cap 120 still provides a lumen 122, but the lumen is open in one embodiment at the distal end instead of providing a beveled opening, which is provided as discussed instead at or near an entrance or proximal end 130 of the cap. Lumen 122 is formed instead to provide rigid sealing cap 120 with length to allow the patient or caregiver to grasp cap 120 from the outside of port tube 80, and along with grasping rigid member holder 160 through port tube 80, twist or torque cap 120 and member holder 160 relative to each other to bend the flexible cylindrical section 144 of flexible sealing member 140, disrupting the beveled seal between cap 120 and flexible sealing member 140, and allowing any of the medical fluids described herein to flow. The medical fluid flowpath described above for flexible plug 10 is the same for flexible plug 110, except that flow through beveled opening 126 occurs after flow through lumen 122. Flexible sealing member 140, rigid member holder 160 and rigid sealing cap 120 may be made of any of the materials discussed herein.

FIGS. 10 and 11 illustrate flexible plug 10 in use with an automated peritoneal dialysis (“APD”) arrangement 90 and a continuous ambulatory peritoneal dialysis (“CAPD”) arrangement 100, respectively. It should be appreciated however that APD arrangement 90 and CAPD arrangement 100 operate equally as well with alternative flexible plug 110 illustrated in connection with FIGS. 8 and 9. FIG. 10 illustrates that APD arrangement 90) includes a PD fluid container 92, such as a flexible bag, which holds a certain amount of a certain type of PD fluid suitable for an APD treatment. A short tube 94 extends is connected to and extends from PD fluid container 92. Port tube 80 of flexible plug assembly 12 is sealed, such as, ultrasonically sealed, heat sealed, or adhered, e.g., solvent bonded, to the inside of short tube 94. Port tube 80 in one embodiment extends from PD fluid container 92 to a patient luer (not illustrated) for connecting to a mating luer of a disposable APD cassette line. Flexible plug assembly 12 includes port tube 80 and flexible plug 10 having sealing cap 20, sealing member 40 and rigid member holder 60 as have been described herein. As discussed herein, sealing cap 20 is sealed to the inside of port tube 80 in one embodiment.

FIG. 11 illustrates that CAPD arrangement 100 includes a PD fluid container 102, such as a flexible bag, which holds a certain amount of a certain type of PD fluid suitable for a CAPD treatment. CAPD arrangement 100 also includes a drain container 104, such as a drain bag. Port tube 80 in an embodiment extends from a short tube connected to PD fluid container 102 to one leg of a Y-connector 108. A drain line 106 in an embodiment extends from a short tube connected to drain container 104 to one leg of a Y-connector 108. Flexible plug 10 including sealing cap 20, sealing member 40 and rigid member holder 60 is located within port tube 80. Sealing cap 20 is again sealed to the inside of port tube 80 in one embodiment.

It should be understood that various changes and modifications to the presently preferred embodiments described herein will be apparent to those skilled in the art. It is therefore intended that any or all of such changes and modifications may be covered by the appended claims. For example, while flexible cylindrical section 44 is illustrated as being cylindrical, the section may have different cross-sectional shapes, such as elliptical or polygonal.

1: A flexible plug for use with a solution container, the flexible plug comprising:
a sealing member including a flexible section that extends to a beveled head;
a rigid member holder secured to the sealing member; and
a rigid sealing cap including a wall defining a beveled opening, the beveled head of the sealing member sealed within the beveled opening to prevent a flow of medical fluid through the flexible plug.

2: The flexible plug of claim 1, wherein the rigid member abuts against the rigid sealing cap.

3: The flexible plug of claim 1, wherein the wall defining the beveled opening is an endwall of the rigid sealing cap.

4. (canceled)

5. (canceled)

6: The flexible plug of claim 1, wherein the beveled opening includes at least one half-round and the beveled head of the sealing member includes at least one mating half-round.

7: The flexible plug of claim 1, wherein the beveled head is formed from the same material as the flexible section or is attached to the flexible section.

8: The flexible plug of claim 1, wherein the rigid member holder includes an annular seat that accepts a mating flange of the sealing member.

9: The flexible plug of claim 8, wherein the flexible section extends from the mating flange of the sealing member through a bore defined by the rigid member holder.

10: The flexible plug of claim 1, wherein the flexible section includes a flexible cylindrical section.

11: A flexible plug assembly for use with a solution container, the flexible plug comprising:
a sealing member including a flexible section that extends to a beveled head;
a rigid member holder secured to the sealing member;
a rigid sealing cap including a wall defining a beveled opening, the beveled head of the sealing member sealed within the beveled opening to prevent a flow of medical fluid through the flexible plug; and
a port tube, sleeve port or Y-connector, at least one of the sealing member, rigid member and rigid sealing cap sealed within the port tube, sleeve port or Y-connector.

12: The flexible plug assembly of claim 11, wherein the port tube, sleeve port or Y-connector is flexible so as to allow a user to grasp and twist or torque the rigid member holder and the rigid sealing cap through the port tube, sleeve port or Y-connector to unseal the beveled head from the beveled opening and to allow the flow of medical fluid through the flexible plug.

13: The flexible plug assembly of claim 11, wherein the rigid member abuts against the rigid sealing cap, the rigid member and rigid sealing cap sealed within the port tube, sleeve port or Y-connector.

14-17. (canceled)

18: A method for manufacturing a medical fluid plug comprising:
overmolding or 2K molding a flexible sealing member and a rigid member holder;
inserting a rigid sealing cap over a flexible section of the flexible sealing member, the rigid sealing cap including a wall defining a beveled opening; and
forming or attaching a beveled head to an end of the flexible section of the flexible sealing member so that the beveled head seals the beveled opening in the wall of the rigid sealing cap.

19: The method of claim 18, which includes thermoforming the beveled head at the end of the flexible section of the flexible sealing member so that the beveled head seals the beveled opening in the wall of the rigid sealing cap.

20: The method of claim 18, which includes adhering the beveled head to the end of the flexible section of the flexible sealing member so that the beveled head seals the beveled opening in the wall of the rigid sealing cap.

21: The method of claim 18, which includes sealing at least the rigid sealing cap within a port tube, sleeve port or Y-connector to form a medical fluid plug assembly.
